(19) United States
(12) Patent Application Publication
(10) Pub. No.: US 2009/0281529 A1
(43) Pub. Date: Nov. 12, 2009

(54) METHOD FOR CONTROLLING A LASER IN THE ABLATION OF THE CORNEAL LAYER OF AN EYE

(76) Inventor: Cesar C. Carriazo, Baranquilla (CO)

Correspondence Address: BARLOW, JOSEPHS & HOLMES, LTD., 101 DYER STREET, 5TH FLOOR, PROVIDENCE, RI, 02903, US

(21) Appl. No.: 11/913,458

(22) PCT Filed: May 2, 2006
(86) PCT No.: PCT/EP2006/004101

§ 371 Date: Aug. 29, 2008

(30) Foreign Application Priority Data
May 2, 2005 (EP) 05009616.3

(51) Int. Cl. A61F 9/01; A61B 18/20

(52) U.S. Cl. 606/5

(57) ABSTRACT

The invention relates to a method for controlling a laser for eye surgery in the excision or ablation of a corneal volume of a human or animal eye, whereby said method comprises the steps of (a) carrying out a pachymetric or pachymetric and topographic measurement of at least one part of the cornea through manual and/or automatic data acquisition by means of a measuring system, (b) calculating a volume element, based on the measured parts of the cornea, using volume-describing functions or by interpolation of the measured data obtained in step a) and/or a following plausibility test of the available data, (c) automatically and/or manually providing and entering data concerning the desired depth, the diameter and the geometry of the corneal excision or ablation, (d) calculating a modified excision or ablation volume, using volume-describing functions and/or by interpolation of the volume element calculated in step b) with the data according to step c), (e) representing the modified excision or ablation volume using volume-describing functions and/or by interpolation of the volume element calculated in step d), (f) automatically and/or manually providing and entering data concerning the correction factors specific to the cornea and to the laser used in the corneal excision or ablation, (g) applying the correction factors acquired in step f) to the excision or ablation volume calculated in step d) and representing the resulting excision or ablation volume, using volume-describing functions, and (i) exporting the data calculated in step g) for external further processing or calculation of a laser spot distribution for generating the excision or ablation volume calculated in step g), taking into consideration device-specific parameters of lasers for eye surgery and exporting or transferring the calculated laser shot coordinates to the laser.

The present invention relates to a method for controlling a laser for eye surgery for the excision or ablation of a defined corneal volume of a human or animal eye, respectively.

Devices and methods for controlling a laser for eye surgery are known. Thus, the international patent application WO 02/22003 describes a device for determining a portion of corneal tissue to be ablated, wherein the volume of the tissue to be ablated is determined with the aid of a pachymetric measurement of the corresponding portion of the cornea to be ablated. Therein, the determined pachymetric data serves for producing a bed inside the cornea for receiving a corresponding donor cornea. Also U.S. Pat. No. 6,551,306 describes a method and a device for controlling the depth ablation on the cornea. Based on topographic/pachymetric data, a corresponding laser is commanded and controlled during surgery.

However, these known methods and devices are disadvantageous in that the known methods calculate and represent the volume to be excised or ablated, respectively, only in insufficient manner. The values for controlling a laser for eye surgery, determined with the known methods, are therefore also insufficient and therefore can only represent an approximation to the optimum volume to be excised or ablated, respectively.

Therefore, it is an object of the present invention to provide a method for controlling a laser for eye surgery for the excision or ablation of a corneal volume of a human or animal eye, respectively, which calculates and represents an excision or ablation volume of the cornea, respectively, optimized for the respective purpose of use, and provides correspondingly optimized control values to the laser.

This object is solved by a method according to the features of claim 1.

Advantageous developments are described in the dependent claims.

A method according to the invention for controlling a laser for eye surgery for the ablation of a corneal layer for example in a patient or donor cornea of a human or animal eye includes the following steps:

(a) pachymetric or pachymetric and topographic measurement of at least one part of the cornea through manual and/or automatic measurement data acquisition by means of a measuring system;
(b) calculating a volume element, based on the measured parts of the cornea, using volume-describing functions or by interpolation of the measured data determined in method step a) and/or a following plausibility test of the available data;
(c) automatically and/or manually providing and entering data concerning the desired depth, the diameter and the geometry of the corneal excision or ablation, respectively;
(d) calculating a modified excision or ablation volume, respectively, using volume-describing functions and/or by interpolation of the volume element calculated in method step b) with the data according to method step c);
(e) representing the modified excision or ablation volume, respectively, using volume-describing functions and/or by interpolation of the volume element calculated in method step d);
(f) automatically and/or manually providing and entering data concerning the correction factors specific to the laser and to the cornea used in the excision or ablation of the cornea, respectively;
(g) applying the correction factors acquired in f) to the excision or ablation volume calculated in d), respectively, and representing the resulting excision or ablation volume, respectively, using volume-describing functions; and
(h) exporting the data calculated in method step g) for external further processing or calculation of a laser spot distribution for generating the excision or ablation volume calculated in method step g), respectively, taking into consideration device-specific parameters of lasers for eye surgery and exporting or transferring the calculated laser shot coordinates to the laser, respectively.

By the use of volume-describing functions or the interpolation of the measured data determined in method step a) an optimized calculation of the volume element of at least one part of the cornea is effected. The corresponding applies to the calculation of the modified excision or ablation volume in d), respectively, by volume-describing functions and/or by interpolation of the volume element calculated in method step b) with the data according to method step c). Volume-describing functions and/or interpolations have distinct advantages over the subtractions of the desired excision or ablation volume, respectively, from the pachymetric determined volume element, used up to now, as for example occurs in WO 02/22003. In contrast to all of the methods known up to now, the method according to the invention uses correction factors specific to cornea and laser for calculating the resulting excision or ablation volume, respectively, which is advantageously additionally post-corrected with the respective device-specific parameters before export to the laser for eye surgery. Thereby it is possible to obtain not only a theoretic, but also an excision or ablation volume optimum in practice, respectively. The correction factors described in method step f) are for example the following factors specific to laser and cornea: spot behavior outside of the focus and fluence, ablation behavior within the stroma, behavior of the stroma upon photodisruption, maximum local repetition frequency, anatomic or stability consideration from topographic and/or pachymetric data, respectively, influences from the storage and the age of the used donor cornea. The device-specific parameters according to method step h) can be the following parameters by way of example: scanner resolution, eye tracking velocity, beam shape, fluence and laser spot size.

In an advantageous development of the method according to the invention, a simulation of the laser ablation for determining the optimum shot distribution with respect to the calculated excision or ablation volume, respectively, is effected before method step (h). Additionally, the volume element and/or the modified excision or ablation volume, respectively, and/or the resulting excision or ablation volume, respectively, can be two-dimensionally or three-dimensionally represented. By the simulation, a determination of the optimum corneal excision or ablation by the laser, respectively, is possible. Furthermore, it is possible that the excision or ablation operation, respectively, can be simulated and observed at each time even before the actual treatment.

In an advantageous development of the method according to the invention, the calculation and the representation of the excision or ablation volume, respectively, is effected according to the method steps b), d), e) and g) by Zernike coefficients, matrices, point coordinates, best fit methods, Cartesian or polar coordinates, vectors or vector coordinates or the like, respectively. By the above-mentioned possibilities of mathematical representation, an extremely exact calculation and representation of the mentioned corneal volumes results.

In another advantageous development of the method according to the invention, the pachymetric measurement of at least one part of the cornea is effected according to method step a) by means of a high-resolution cornea thickness measuring device such as for example a Scheimpflug system, Orbscan, OCT, Astramax topography measuring device or an ultrasonic-based, full-area measuring system such as the Artemis. It is also possible to use a one-point ultrasonic pachymeter with ultrasonic measuring head. Therein, the pachymetric measurement includes a plurality of measurement points on the cornea.

In another advantageous development of the method according to the invention, the determination of the optimum excision or ablation volume, respectively, is effected by applying pachymetric to the topographic data of the cornea (pachymetric/topographic laser correction). From the prior art, the sole use of topographic data for determining the ablation volume without simultaneous consideration of the respective pachymetry is known. The combination of pachymetric and topographic data allows substantially more accurate consideration of the effect of locally different pachymetric values on the local topography, and thus avoids a too great local stability weakening of the cornea and a keratoconus formation (corneal curvature) associated therewith.

The pachymetric measurement of the cornea and the calculation of the volume element according to the method steps a) and b) especially serve for determining defective visions of the eye or for determining a graft bed for a corneal graft or for processing a corneal graft.

Further advantages, details and features of the method according to the invention are exemplarily illustrated by way of two flow diagrams presented in the figures. There show

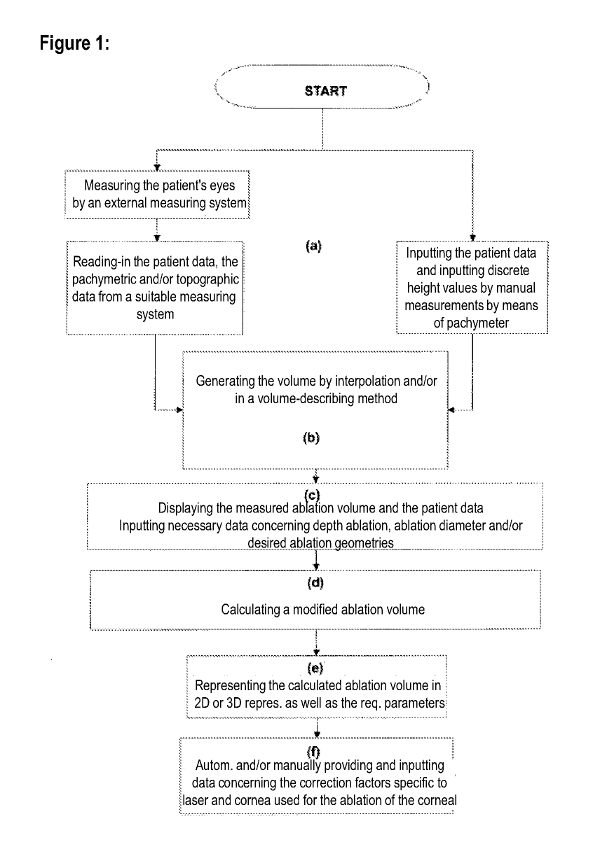

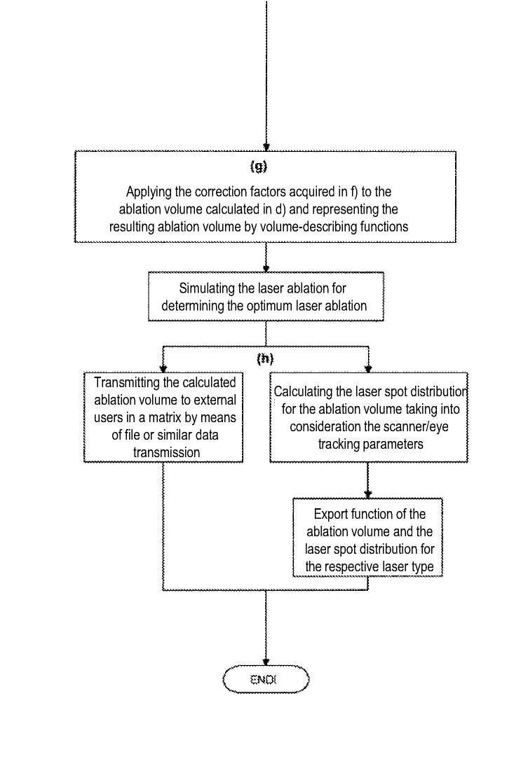

FIG. 1 a flow diagram of the method according to the invention for controlling a laser for eye surgery in the treatment of a patient's cornea; and

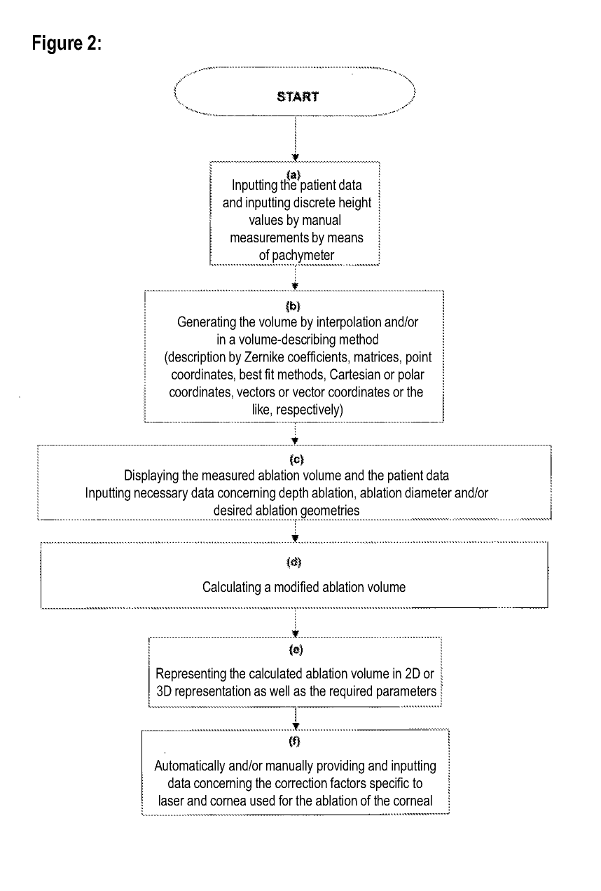

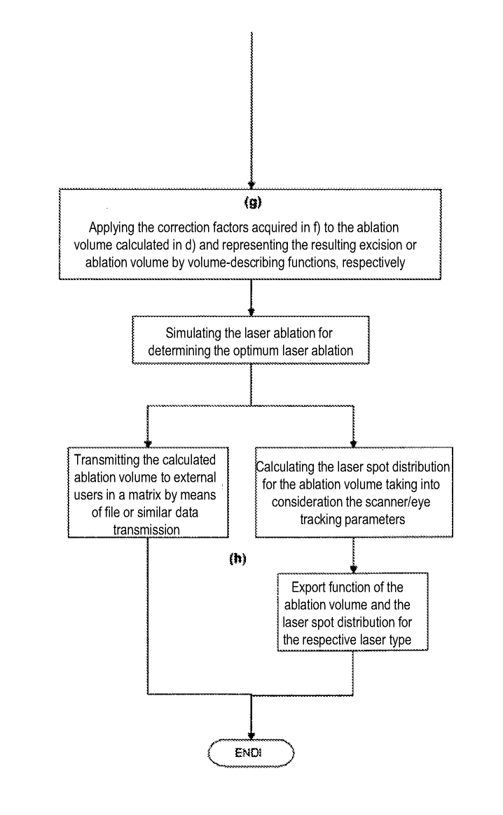

FIG. 2 a flow diagram of the method according to the invention for controlling a laser for eye surgery in the treatment of a donor cornea.

FIG. 1 shows a flow diagram of a method for controlling a laser for eye surgery in the treatment of a patient's cornea. Therein, the laser can be an excimer laser, a solid state laser, an fs laser, a ps laser or very generally a cutting laser or ablation laser, respectively. One recognizes that in a first method step a), besides the pachymetric or pachymetric and topographic measurement of at least one part of the cornea, the corresponding data can be made available either manually or by reading-in from a diagnostic system.

In the following method step b), the calculation and representation of the volume element measured in a) is effected by interpolation or volume-describing functions.

Within the scope of the method step c), required data such as desired depth, diameter and geometry of the excision or ablation are inputted, respectively.

In the following method step d), the calculation of the excision or ablation volume, respectively, is effected, taking into consideration the values input in c) by volume-describing functions and/or by interpolation.

In method step e), the representation of the modified excision or ablation volume, respectively, by volume-describing functions in 2D or 3D and/or interpolation is effected.

In method step f), the automatic and/or manual provision and input of data with respect to the laser used for the excision or ablation of the cornea, respectively, especially the provision and input of correction factors specific to laser and cornea, is effected.

In the following method step g), the calculation and display of a resulting excision or ablation volume, respectively, is effected by application of the correction factors specific to laser and cornea from method step f) to the theoretical excision or ablation volume from method step d), respectively.

Before the completing method step (h), according to this embodiment, a simulation of the corneal excision or ablation, respectively, is performed for verifying the optimum, laser-specific excision or ablation behavior, respectively, before the actual corneal excision or ablation, respectively.

Finally, in the concluding method step (h), an export of the calculated excision or ablation volume, respectively, to external users for external further processing or a direct calculation of the data provided in method step g) for calculating the laser shot coordinates is effected, taking into consideration the device-specific parameters such as for example eye tracking or scanner parameters, respectively. Different laser shot profiles such as for example cutting contour or layered volume ablation, volume-describing and cutting contour describing functions are also considered. Finally, provision of an export function for the calculated excision or ablation volume, respectively, and of the laser shot coordinates for the respective laser type results from this.

FIG. 2 shows a flow diagram of a method for controlling a laser for eye surgery in the treatment of a donor cornea. One recognizes that in contrast to the embodiment illustrated in FIG. 1, the specific patient data and especially the input of discrete height values are effected by manual measurement by means of a pachymeter. The further method steps correspond to those described in the first embodiment.

1-7. (canceled)

9. A method for controlling a laser for eye surgery for the excision or ablation of a corneal volume of a human or animal eye, respectively, wherein the method includes the following steps:
(a) pachymetric or pachymetric and topographic measurement of at least one part of the cornea through manual and/or automatic measurement data acquisition by means of a measuring system;
(b) calculating a volume element, based on the measured parts of the cornea, using volume-describing functions or by interpolation of the measured data determined in method step a) and/or a following plausibility test of the available data;
(c) automatically and/or manually providing and entering data concerning the desired depth, the diameter and the geometry of the corneal excision or ablation, respectively;
(d) calculating a modified excision or ablation volume, respectively, using volume-describing functions and/or by interpolation of the volume element calculated in method step b) with the data according to method step c);
(e) automatically and/or manually representing the modified excision or ablation volume, respectively, using volume-describing functions and/or by interpolation of the volume element calculated in method step d);
(f) providing and entering data concerning the correction factors specific to the laser and to the cornea used for the excision or ablation of the cornea, respectively;
(g) applying the correction factors acquired in f) to the excision or ablation volume calculated in d), respectively, and representing the resulting excision or ablation volume, respectively, using volume-describing functions; and
(h) exporting the data calculated in method step g) for external further processing or calculation of a laser spot distribution for generating the excision or ablation volume calculated in method step g), respectively, taking into consideration device-specific parameters of lasers for eye surgery and exporting or transferring the calculated laser shot coordinates to the laser, respectively.

10. The method according to claim 9, characterized in that:
a simulation of the laser ablation for determining the optimum shot distribution with respect to the calculated excision or ablation volume, respectively, is performed before method step (h).

11. The method according to claim 9, characterized in that:
the calculation or representation of the determined excision or ablation volume, respectively, according to the method steps b), d), e) and g) by volume-describing functions is effected by Zernike coefficients, matrices, point coordinates, best fit methods, Cartesian or polar coordinates, vectors or vector coordinates, respectively.

12. The method according to claim 9, characterized in that:
the pachymetric measurement of at least one part of the cornea according to method step a) is effected by means of a cornea thickness measuring device.

13. The method according to claim 9, characterized in that:
the pachymetric measurement of at least one part of the cornea is effected by the determination and measurement of the topography and/or pachymetry of the corneal surface.

14. The method according to claim 9, characterized in that:
the pachymetric measurement of at least one part of the cornea includes a plurality of measurement points on the cornea.

15. The method according to claim 9, characterized in that:
the pachymetric measurement of the cornea and the calculation of the volume element according to method steps a) and b) serves for determining defective visions of the eye or for determining a graft bed for a corneal graft or for processing a corneal graft.

16. The method according to claim 9, characterized in that:
the calculated volume elements can be represented two-dimensionally and/or three-dimensionally.
